(19) United States
(12) Patent Application Publication
(10) Pub. No.: US 2016/0235314 A1
(43) Pub. Date: Aug. 18, 2016

(54) BIOSENSOR DEVICE, SYSTEMS AND METHODS THEREOF

(71) Applicant: National Cheng Kung University, Tainan City (TW)

(72) Inventors: LIANG-MIIN TSAI, Tainan City (TW); FAN-MING YU, Tainan City (TW); CHOU-CHING LIN, Tainan City (TW); JU-YI CHEN, Tainan City (TW); HUI-WEN YANG, Tainan City (TW); KUAN-JUNG LI, Tainan City (TW)

(73) Assignee: National Cheng Kung University, Tainan City (TW)

(21) Appl. No.: 15/016,419

(22) Filed: Feb. 5, 2016

Related U.S. Application Data
(60) Provisional application No. 62/116,938, filed on Feb. 17, 2015.

(51) Int. Cl. A61B 5/024; A61B 7/04; A61B 5/02; A61B 5/046; A61B 5/021; A61B 5/00

(52) U.S. Cl. CPC A61B 5/02444; A61B 5/02438; A61B 5/021; A61B 5/725; A61B 5/7239; A61B 5/02028; A61B 5/046; A61B 7/04

(57) ABSTRACT

The present disclosure relates to devices and methods for sensing ACVG. In one example, the device comprises an ACVG sensor for sensing signals of heart beat and arterial pulse in a predetermined period. The ACVG sensor transforms the signals to electrical output. The analog-to-digital converter receives the electrical output and converts the electrical output into digital signals. The present disclosure further relates to methods of determining physiological conditions. In one example, the method comprises receiving an ACVG, providing a waveform data by processing the ACVG, extracting at least one data point from a predetermined time interval of the waveform data, obtaining indicators based on the at least one data point, and determining a physiological condition according to the indicator.

### FIELD

The subject matter herein generally relates to an audiocardiovasculography (ACVG) sensor to non-invasively monitor heart rhythm and hemodynamics of a subject.

### BACKGROUND

Clinically, arrhythmias can be diagnosed simply by a 12-Lead electrocardiograph (ECG) System, or by long-term data obtained from Holter ECG monitor. The two diagnostic systems are currently industrial standard for arrhythmias detection. To obtain an ECG data, a test subject needs to lie down and allow all electrode pads to be installed only to obtain data of several seconds. It takes much longer and is even more complicated to use Holter ECG monitor than a standard ECG system. While wearing the Holter monitor, the test subject cannot take showers and needs to install multiple electrode pads, which is inconvenient for the test subject as well as for the physicians. In addition, current devices for arrhythmias examination and blood pressure monitoring are also inconvenient clinically and are sometimes invasive which might lead to bleeding and infection. The test subjects are also required to stay still for a long time either for installation or for monitoring.

### BRIEF DESCRIPTION OF THE DRAWINGS

Implementations of the present technology will now be described, by way of example only, with reference to the attached figures.

FIG. 1A is a cross-sectional view of one of the example of acoustic sensors.

FIG. 1B is audiocardiovasculograms obtained from different sensing devices.

FIG. 2 is a flow chart of a method in accordance with the present disclosure.

FIG. 3 illustrates a block diagram of the functioning of one of the example of a status monitoring system with at least one ACVG sensor.

FIG. 4A is a flow chart of a method in accordance with the present disclosure.

FIG. 4B is a typical audiocardiovasculogram derived from an acoustic sensor described in FIG. 1A.

FIG. 5 illustrates a scatter diagram derived from an audiocardiovasculography of a healthy person.

FIG. 6 illustrates synchronized electrocardiogram and audiocardiovasculogram of a patient with ventricular premature contractions.

FIG. 7 illustrates a scatter diagram derived from an audiocardiovasculography of a pacemaker-dependent patient.

FIG. 8 illustrates a scatter diagram derived from an audiocardiovasculography of a patient with atrial fibrillation.

FIG. 9A illustrates a scatter diagram derived from an audiocardiovasculography of a patient with atrial flutter.

FIG. 9B illustrates a scatter diagram derived from an audiocardiovasculography of a patient with atrial premature contractions.

FIG. 9C illustrates a scatter diagram derived from an audiocardiovasculography of a patient with ventricular premature contractions.

FIG. 10 illustrates a scatter diagram of the relationships between a feature extracted from an audiocardiovasculography and an ECG and diastolic blood pressure.

FIG. 11 illustrates a boxplot of patients with complete right bundle branch block (CLBBB), patients with complete right bundle branch block (CRBBB), and healthy individuals (Control).

FIG. 12 illustrates a curve diagram of the relationships between systolic ejection time and value of ejection fraction of left ventricle.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. The drawings are not necessarily to scale and the proportions of certain parts may be exaggerated to better illustrate details and features. The description is not to be considered as limiting the scope of the examples described herein.

Several definitions that apply throughout this disclosure will now be presented.

The term “coupled” is defined as connected, whether directly or indirectly through intervening components, and is not necessarily limited to physical connections. The term “configured” is defined as arranged or designed so as to fit it for a designated task. The connection can be such that the objects are permanently connected or releasably connected. The term “comprising,” when utilized, means “including, but not necessarily limited to”; it specifically indicates open-ended inclusion or membership in the so-described combination, group, series and the like. The term “nearly equal” when utilized, means “highly similar but not necessarily identical”.

The present disclosure is described in relation to an ACVG sensing device configured to sensing physiological conditions.

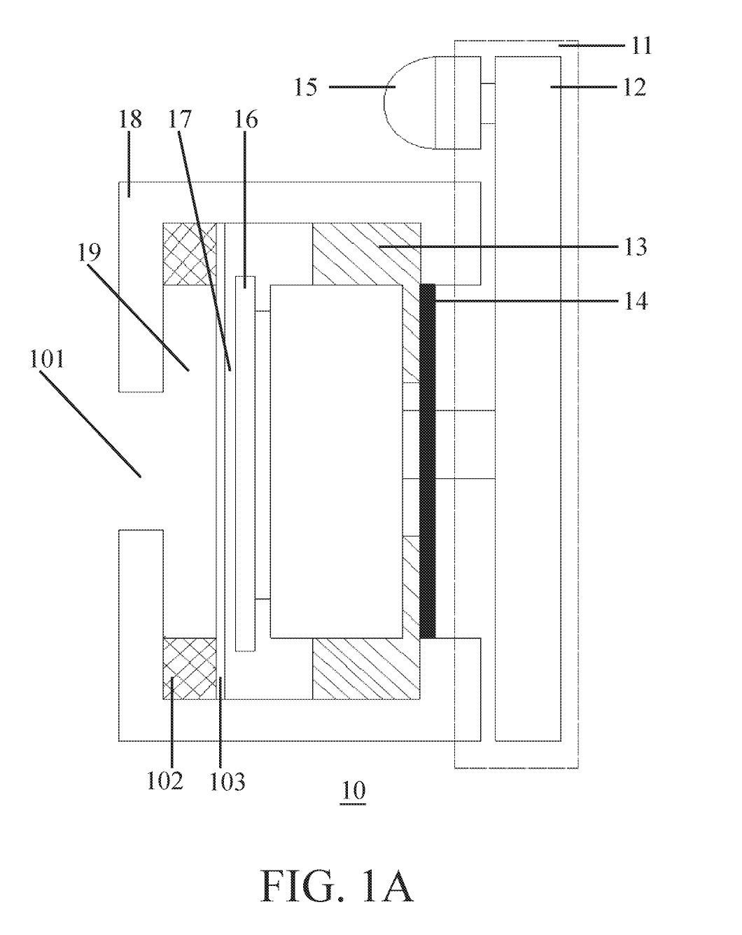

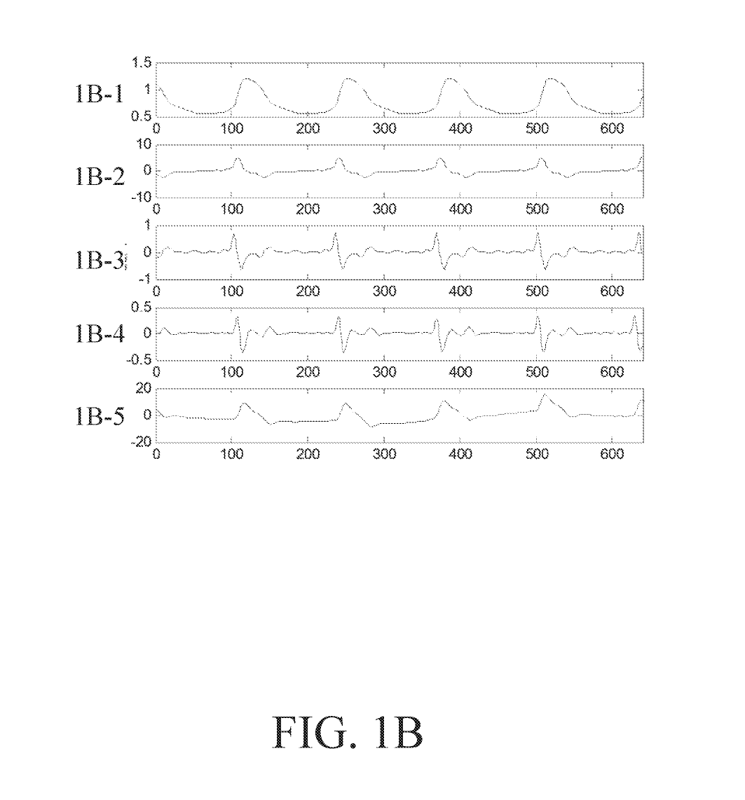

The ACVG sensing device comprises at least one ACVG sensor. In one example the ACVG sensor is an acoustic sensor. FIG. 1A shows a cross-sectional view of one of the example of acoustic sensors. An acoustic sensor 10 is built on a fixation assembly 11 which can be a belt, a watch, a wrist band or other devices that are capable of fixing the acoustic sensor onto a target position. Built in the fixation assembly 11 there can also be a positioning module 15 used to search a preferable target position. The target position, for example, can be a skin surface over the desired blood vessel. The positioning module 15 can be but is not limited to an infrared sensor which is capable of indicating superficial blood vessels. It is to be noted that the positioning module 15 can be a separate module from the acoustic sensor or can be a built-in module in the acoustic sensor 10 or in the fixation assembly 11. The acoustic sensor 10 comprises at least an amplifier 12, a printed circuit board 14, a holder assembly 13, a back plate 16, a spacer 17, a diaphragm 103, a ring 102, a housing 18, a medium cavity 19 and a target position-aiming region 101. The housing 18 is configured to hold at least the printed circuit board 14, the holder assembly 13, the back plate 16, the spacer 17, the diaphragm 103 and the ring 102 inside and to fix all parts directly or indirectly to the amplifier 12 and/or the fixation assembly 11. The housing 18 is configured to hold the holder assembly 13. The holder assembly 13 is configured to fix the back plate 16 to a position. The diaphragm 103 is fixed either by the housing 18 or by the holder assembly 13 (not shown). Inside the housing 18, the back plate 16 is mounted directly or indirectly on the holder assembly 13. Between the back plate 16 and the diaphragm 103 is the spacer 17. Opposite to the spacer 17, the other side of the diaphragm 103 is a medium cavity 19. The medium used here in this example can be air for which acoustic waves are transmitted through. On one side of the housing 18 opposite to the fixation assembly 11 is the target position-aiming region 101. When in use, after the positioning module 15 identifies a target position, the acoustic sensor 10 is moved to the position and the target position-aiming region 101 would cover or circle the target position. Together the target position (for example the skin on top of the radial blood vessel), the housing 18 and the diaphragm 103 defines therein the medium cavity 19, which when in use is an enclosed cavity. Alternatively, in order to increase the medium volume and for other mechanical purposes, there can also be a ring, for example an o-ring, with a predetermined thickness so as to enlarge one stroke of the space and thus increase the medium volume. In this example, the ring 102 is shown to increase the volume of the medium cavity 19, fix the diaphragm 103 to the interior of the housing 18 and allow the overall design to be mechanically possible. The diaphragm 103 and the back plate 16 are two electrodes electrically coupled to the printed circuit board 14. When in use, an auditory pressure is transmitted through the enclosed medium cavity 19 and cause vibrating events on the diaphragm 103. The vibration of the diaphragm 103 changes the distance (the spacer 17) between the diaphragm 103 and the back plate 16, leading to a capacitance change. The capacitance change further creates a change in voltage, which forms an electrical signal sent via wire establishment to the amplifier 12 then to the acoustic sensor 10 output.

As shown in FIG. 1A, such acoustic sensor 10 has at least an enclosed cavity when in use to receive a desired signal from acoustic vibration and to exclude unwanted environmental noise. Such acoustic sensor 10 has a response frequency including a range from approximately at 0.5 hertz to 1000 hertz. The acoustic sensor 10 can also comprise wired or wireless signal transduction means to send the microphone output to an external device. In this example, there is only one diaphragm 103 used in the condenser microphone. It is to be noted that various transforms of the present disclosure that are capable of obtaining either directly or indirectly an auditory signal of heart beats and arterial pulse, can be practiced without specific details. For example, the acoustic sensor 10 can also have multiple diaphragms if it is capable of obtaining an auditory signal of heart beats and arterial pulse.

The acoustic sensor 10 is configured to receive hemodynamic signals and auditory signals (signals of heat beats and arterial pulse) derived from the sound generated by the blood flowing through a vessel. The blood flows from the heart, to arteries, veins, and various kinds of blood vessels such as capillaries and back to the heart again. The circulation is generally regarded as an enclosed flow circulation system, where the vibration and the auditory signal generated at any position of the blood circulation system is detectable via the acoustic sensor 10 described herein. The acoustic signal contains all changes of hemodynamic pressure signal of the cardiovascular system, which not only represent the heart rhythm but also the mechanical function of the heart. Therefore the acoustic signal reflects the dynamic pressure changes of a hemodynamic system of a human subject and can further indicate cardiovascular diseases of the subject. Such signals are ambient yet can be physically amplified by the acoustic sensor 10 described herein through the enclosed cavity design and through positioning the acoustic sensor 10 at the skin surface over the desired blood vessel, such as at the wrist over radial artery.

In the present disclosure, the ACVG sensor comprises at least a transducer which can transduce physiological signals into electrical output. Specifically, the physiological signals are signals of heart beat and arterial pulse. The ACVG sensor may further comprise other functional modules, for example, an analog differentiator.

FIG. 1B shows audiocardiovasculograms obtained from different audiocardiovasculographis (ACVGs) sensed by ACVG sensing devices, wherein ACVG means the signals of heart beats and arterial pulse sensed in a predetermined period; wherein the audiocardiovasculogram is a sound waveform diagram derived from a kind of ACVG. Performantly, ACVG in the present disclosure can be C-ACVG, PE-ACVG, or P-ACVG. 1B-1 shows an audiocardiovasculogram obtained by a blood pressure monitor. The signal received by a blood pressure monitor is called Pressure-audiocardiovasculography (P-ACVG), which was plotted in 1B-1. The measurement of the horizontal axis of 1B-1 is an amplitude derived from a blood pressure monitor. As shown in FIG. 1B, re-calculating the 1B-1 by differentiating y (amplitude) by x (time) would give 1B-2. The term “differentiating” means “to undergo a process of mathematic calculation of finding a derivate”. 1B-2 is nearly equal to an audiocardiovasculogram obtained by a piezoelectric microphone. The signal received by a piezoelectric microphone is called Piezoelectric-audiocardiovasculography (PE-ACVG). Re-calculating the 1B-2 by differentiating y of 1B-2 by x (time) would give 1B-3. In order words, a waveform data similar with 1B-3 can be obtained by calculating the first order derivative from PE-ACVG or by calculating the second order derivative from P-ACVG. 1B-3 is nearly equal to an audiocardiovasculogram obtained by a capacitive microphone. The signal received by a capacitive microphone is called capacitive-audiocardiovasculography (C-ACVG). Performantly, the capacitive microphone is an acoustic sensor described in FIG. 1A. 1B-4 shows an audiocardiovasculogram obtained by an acoustic sensor described in FIG. 1A. The measurement of the vertical axis of 1B-4 is an amplitude derived from the acoustic sensor described in FIG. 1A. As shown in 1B-4, the pattern is nearly equal to 1B-3. The present disclosure herein is that the auditory signals of heart beats and arterial pulse sensed in a predetermined period and outputted by a capacitance microphone (C-ACVG) can derive from a blood pressure monitor and a piezoelectric microphone with a kind of mathematical transformation. To verify such calculation, 1B-4 is integrated twice and gives 1B-5. As shown in 1B-5, the pattern is nearly equal to 1B-1. The differentiation as described above can be achieved by a differentiator or a conductive medium, and wherein the differentiator may be an analog differentiator or a digital differentiator. In one example, the digital differentiator is a processor which has a differentiating calculating function. In another example, the analog differentiator is a differentiating circuit coupled with the transducer. The conductive medium may be a gel with a predetermined damping factor or an air column.

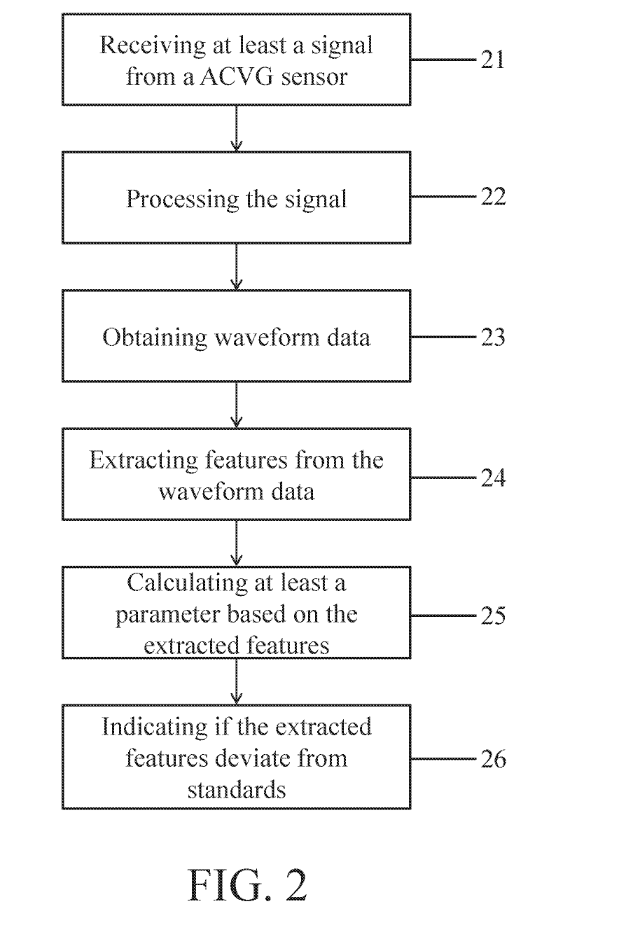

FIG. 2 is a flow chart of a method in accordance with the present disclosure. Different kind of ACVG sensors are used in this example. The ACVG sensor in the present disclosure can be but is not limited to a microphone, a capacitive microphone, a piezoelectric microphone or blood pressure monitor so long as the ACVG sensor is capable of obtaining either directly or indirectly an ACVG. After the ACVG sensor is equipped on a subject over the target position, the ACVG sensor will be activated and start transmitting at least a signal to a processor, as shown in FIG. 2 Step 21. The signal can be a single point signal or can be a signal series with multiple data points. Specifically, the signal is an ACVG. The received signal is then processed as in Step 22. The processing events can be a one-time event in different order or in any combination with multiple events in any possible orders. The end product of processing the signals is then outputted in a form of waveform data as in Step 23. Specifically, the waveform data can be a sound waveform data. The waveform data is then further processed by one or a set of predetermined parameter as in Step 24, such as the peak value, amplitude, ordinary frequency (the number of oscillations that occur each second of time), angular frequency, wavelength, phase or a mathematical calculation using related parameters. For example, a data point of peak amplitude can be extracted from the waveform data by identifying a peak Y value (amplitude) in a predetermined time interval, for instance, 0.5 second, and identifying the X value (time) of the corresponding data point. With multiple Ys and their corresponding Xs, a series is extracted from the waveform data. Features obtained from the one or set of parameters and extracted from the waveform data (Step 24) are used to indicate certain physiological conditions (Step 26), such as cardiovascular status, arrhythmias, pacemaker-dependent condition, atrial premature contraction, atrial fibrillation, atrial flutter, pulse deficit, or ejection fraction. The method may further comprise sending informing signal to the user of the ACVG sensor to notify an abnormal or a healthy status. Such indication can identify a deviation from standards (Step 26) within a subject or a predetermined standard database.

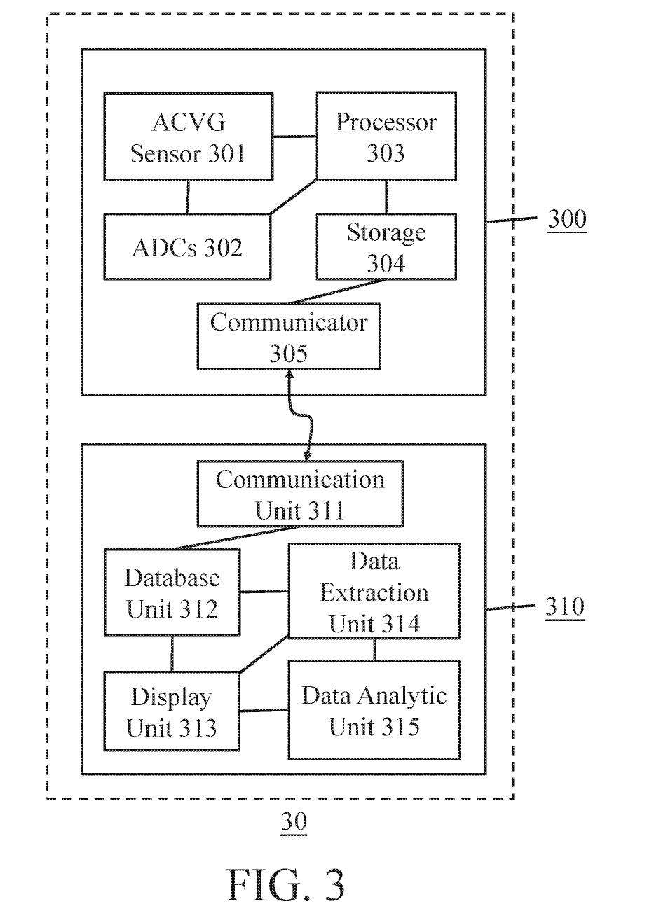

FIG. 3 shows a functional block diagram of one of the example of present disclosure. A status monitoring system 30 which utilize at least one ACVG sensor 301. The status monitoring system 30 contains two separated parts, a sensing device 300 and an analyzing device 310.

A status monitoring system 30 for monitoring and indicating physiological and/or cardiovascular conditions of a subject and for providing audible and/or visual indications to a user is illustrated in FIG. 3. Generally, the sensing device 300 comprises at least one ACVG sensor 301, at least one analog-to-digital converter (ADC 302) and a processing system including a processor 303 (may be a central processing unit, CPU) and/or a digital signal processor (not shown). The ACVG sensor 301 will sense signals and receive electrical output, for instance, voltage and current, of the incoming lines from an electrical power distribution system, for instance, an electrical circuit. The voltage output of the ACVG sensors 301 will be converted to a digital signal by the ADC 302. The processor 303 then operates on the digital signal to generate an information output signal which varies in relation to the voltage component of the analog input signal. Once the signal enters the processor 303, the storage 304 is accessed for its instructions. A power supply is also configured to provide power to the components of the sensing device 300 (not shown).

The storage 304 is configured to store the files including signal filtering, signal processing, visual and/or audible instructions. The storage 304 may be internal storage memory, for instance, random access memory (RAM), or removable memory such as magnetic storage memory; optical storage memory, for instance, the various known types of CD and DVD media; solid-state storage memory, for instance, a CompactFlash card, a Memory Stick, SmartMedia card, MultiMediaCard (MMC), SD (Secure Digital) memory; or any other memory storage that exists currently or will exist in the future.

The signal saved in storage 304 will be transferred from a communicator 305 to an analyzing device 310 by wired or wireless signal transmission means. The signal transduction means can be but not limited to USB, Bluetooth, ZigBee, RFID, and Wi-Fi, etc.

In present disclosure, the ACVG sensor 301, the ADC 302, the processor 303, the storage 304, and the communicator 305, as shown in FIG. 3, are not necessarily present in a single device.

Generally, analyzing device 310 comprises a communication unit 311, a database unit 312, a display unit 313, a data extraction unit 314, and a data analytic unit 315. The communication unit 311 can communicate with communicator 305 of sensing device 300 and receives signal from sensing device 300. The received signal can be saved in the database unit 312. The database unit 312 also stores a reference database which contains typical data of particular physiological statuses. The data extraction unit 314 can extracts specific parameters from signal received by communication unit 311. The data analytic unit 315 compares the extracted parameter set from the receiving signal and reference database to find out the most relevant physiological status. The display unit 313 presents a user interface which allows user to easily operate the communication unit 311, the database unit 312, the data extraction unit 314, and the data analytic unit 315.

In present disclosure, the communication unit 311, the database unit 312, display unit 313, the data extraction unit 314, and the data analytic unit 315, as shown in FIG. 3, are not necessarily present in a single device.

In another example, all elements within the sensing device 300 and the analyzing device 310 can be built in a single device.

In present disclosure, an ACVG sensing device refers to any kind of combination comprises an ACVG and an ADC, for example, an ACVG sensing device may be the sensing device 300 or the status monitoring system 30 in FIG. 3.

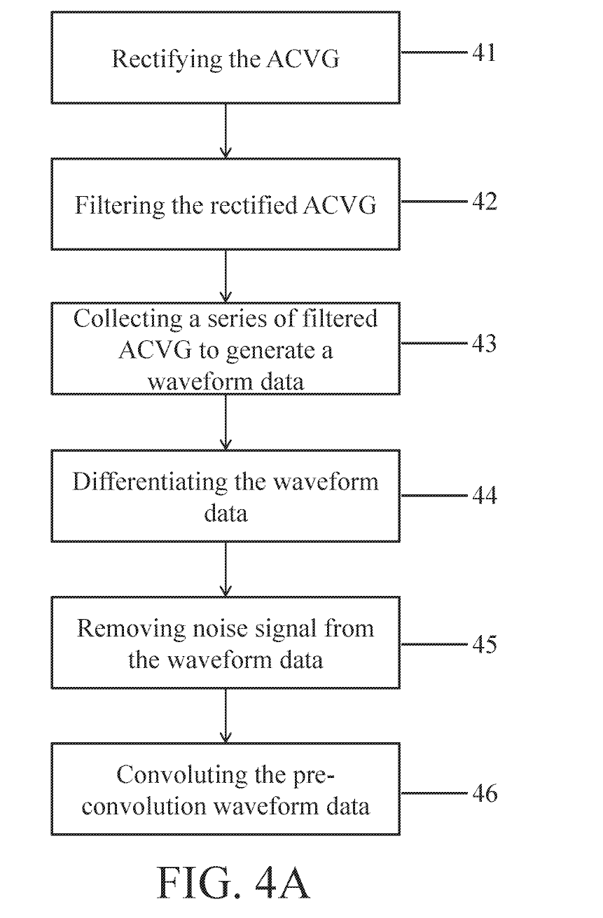

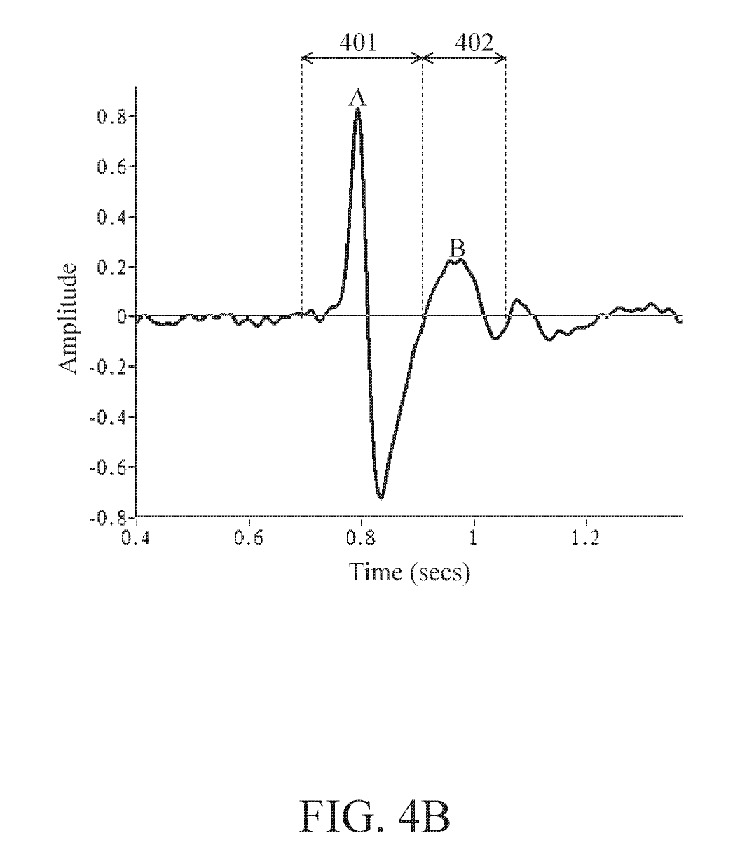

FIG. 4A is a flow chart of a method in accordance with the present disclosure. A received signal is filtered and processed as disclosed in FIG. 2 Step 22. In this example, Step 22 is further elaborated without limitation to the present invention. A signal is received from an ACVG sensor. Specifically, the signal is an ACVG. The received signal is then rectified by converting data with negative into zero or a positive corresponding value as in Step 41. For example, the values of a series of received signals [1, 0, −3, 2] will be rectified into [1, 0, 0, 2] or [1, 0, 3, 2]. The rectification can be a single phase rectification, half-wave rectification, full wave rectification or other kinds of rectification. In one example, the rectification of the received signal is processed by entering the value of y measurement (Y) into a function such as (Y+abs(Y))/2, wherein abs(Y) is the absolute value of Y value. As in Step 42, the rectified ACVG is further filtered through a bandpass filter to obtain a filtered ACVG with a desirable frequency range. Collecting a series of filtered ACVG can generate a waveform data (Step 43). In this example, the waveform data is a sound waveform data. In one example, the bandpass filter is a zero phase shift bandpass filter and allows signals with response frequency between 5 hertz to 35 hertz to pass. In this example, the desirable signals within a range of response frequency at 5 hertz to 35 hertz are the filtered ACVGs. As in Step 44, the waveform data can be further differentiated by obtaining a difference of Y value between adjacent X (time). The differentiated waveform data is then filtered to remove any signal frequency (the noise signal) above 30 hertz and allows a pre-convolution waveform data to pass. The pre-convolution waveform data is a noise-free or noise-reduced data which is ready to be further analyzed (Step 45). In one example, the further analysis is to obtain a peak Y value with a corresponding X value within a predetermined time interval. In this example, the peak Y value can be defined by convoluting the pre-convolution waveform data with a predetermined function (Step 46). Performantly, the predetermined function may be a constant function with a limited input value. For example, the predetermined function is f(x)=1 and the limited input is the integers between 1 to 10, therefore the output of the predetermined function is [1, 1, 1, 1, 1, 1, 1, 1, 1, 1]. In this disclosure, the predetermined function is not necessarily a constant function.

FIG. 4B is a typical audiocardiovasculogram which is a sound waveform diagram derived from an acoustic sensor described in FIG. 1A. As shown in FIG. 4B, there are two waves in the predetermined time interval, a first wave 401 and a second wave 402. The first wave 401 has a peak Y value which represents a data point denoted as A. The second wave 402 has a smaller peak Y value which represents a data point denoted as B.

The present disclosure is an ACVG sensing device which is used for diagnostic, monitoring and indicating cardiovascular status of a subject. The ACVG sensing device in present disclosure is also used for continuously monitoring the hemodynamic signal and the corresponding ACVG of a subject over a period of time. The present disclosure also relates to a combination of ACVG, and electrocardiography (ECG) signals received by an ACVG sensor and an ECG probe to diagnose, examine, monitor and indicate cardiovascular status of a subject. The ECG probe may also be a device, a set of leads, or other modules for receiving ECG signals. The cardiovascular status described herein comprises but not limited to pacemaker-dependent status, atrial premature contraction (APC), atrial fibrillation, atrial flutter, pulse deficit, complete left bundle branch block (CLBBB), ejection fraction (EF) and blood pressure.

Experiment Procedure

Each of the following examples is experimented by the same set of base protocol. Around 100 human subjects are selected from healthy individuals, patients with known arrhythmia including VPC, APC, atrial fibrillation, atrial flutter, CLBBB, heart failure and patients that are completely dependent on pacemakers. Subjects were seated and told to relax without any movement. The ACVG sensing device, comprises an acoustic sensor described in FIG. 1A, was wore and fixed on the right wrist where the aperture of the acoustic sensor (as illustrated in FIG. 1A target position-aiming region 101) covers the skin surface of the radial artery. For experiments of using both the ACVG sensing device and the ECG probe, the ACVG sensing device is equipped as previously described and the electrodes of ECG probe were equipped on the right hand, left and right leg as standard practice. Both the ACVG sensing device and the ECG probe were set to synchronize analog-to-digital conversion and synchronize the sampling rate. In one example, both the ACVG sensing device and the ECG probe have a sampling rate of 200 hertz. In another example, the ACVG sensing device and the ECG probe can have a sampling rate above 200 hertz, and the received signals may be processed by down sampling.

I. Healthy Subject

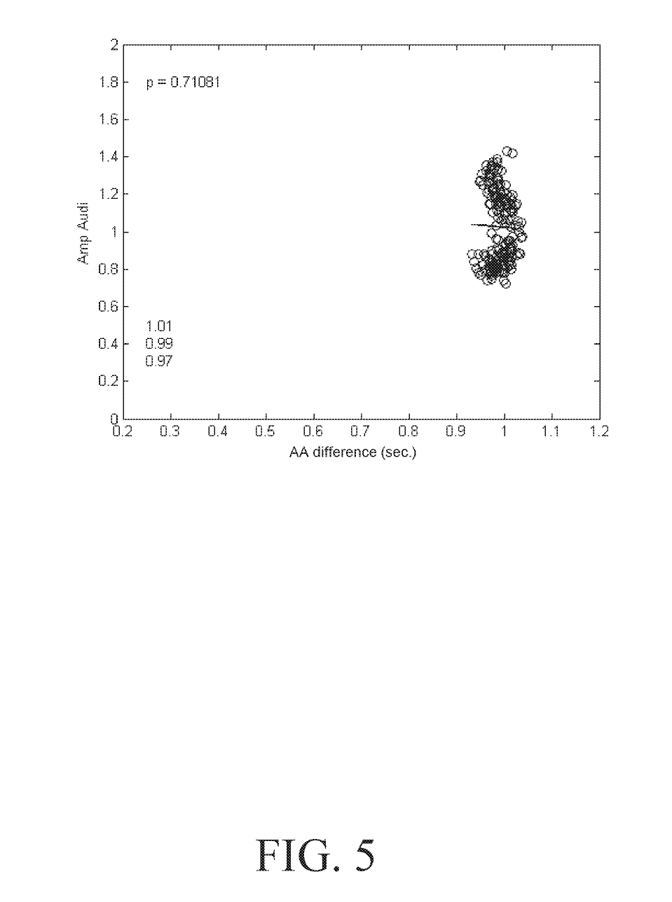

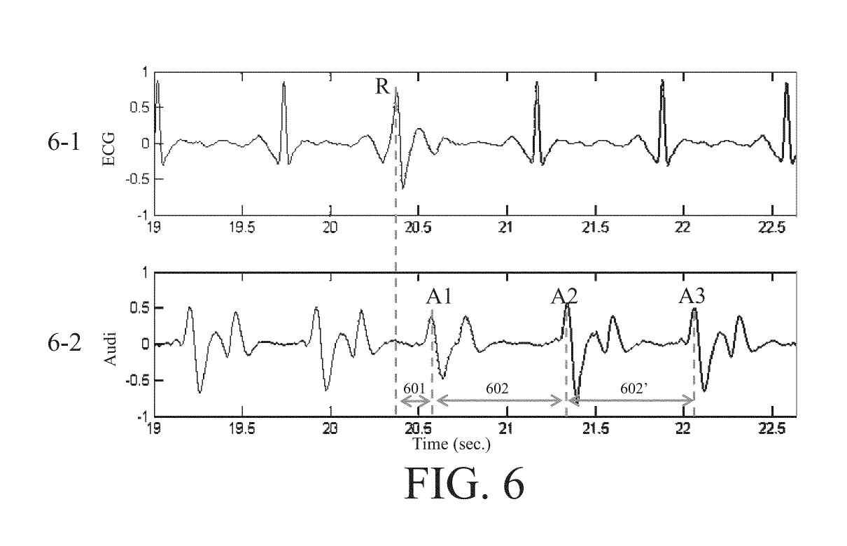

FIG. 5 shows a scatter diagram which indicates a healthy individual. The measurement of horizontal axis of the scatter diagram is the AA difference. AA difference is the interval of the corresponding data points between two adjacent waveform. Specifically, the corresponding data points are two A points, for example, A1, A2 and A2, A3 in FIG. 6, and the AA differences are intervals between two adjacent A points, for example, the interval 602 and 602′ in FIG. 6. Each A point is determined by identifying the point with peak Y value within 0.5 second. In another example, the corresponding data points between two adjacent waveform can be two adjacent B points (not shown). The measurement of Amp Audi of vertical axis in FIG. 5 is defined by each peak Y value in the first wave, obtained by the ACVG sensing devices, in a predetermined time interval, for instance, 0.5 second. This feature (AA difference) is extracted from the waveform data as described in FIG. 2. A healthy individual would show a constant AA difference with a moderate dispersion of Amp Audi because the heart rate is rather regular and consistent but the strength of the heart beats might vary slightly. Therefore, a centralized data points in terms of AA difference and moderately dispersed Amp Audi in a scatter diagram would indicate a healthy heart condition in terms of heart rhythm.

II. Pacemaker-Dependent Status

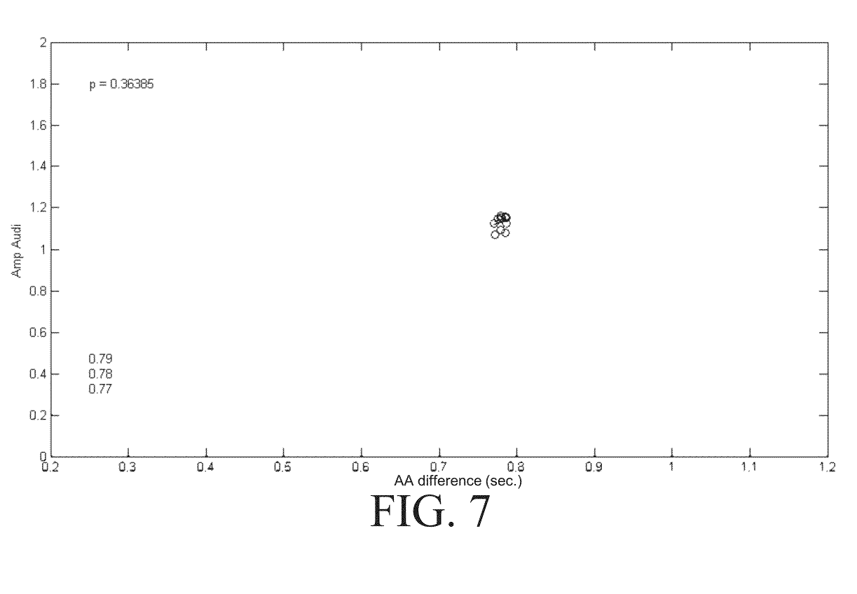

FIG. 7 shows a scatter diagram which indicates a pacemaker-dependent patient. The measurement of horizontal axis of the scatter diagram is AA difference. A patient under certain conditions may need pacemaker installing. Instead of relying on the normal heart function, the patient usually partially or completely depends on the pacemaker to send out the heartbeat signal. A patient who is dependent on the pacemaker was examined. As shown in FIG. 7, the patient would show a constant AA difference with a consistent Amp Audi because the heart rate is very regular and consistent and the pulse volume of heart beats is also consistent. This is because the heartbeats are regulated by the pacemaker; the frequency and the signal intensity are highly consistent. Therefore, a centralized data points in terms of AA difference and Amp Audi in a scatter diagram would indicate that a patient completely depends on the pacemaker.

III. Atrial Fibrillation

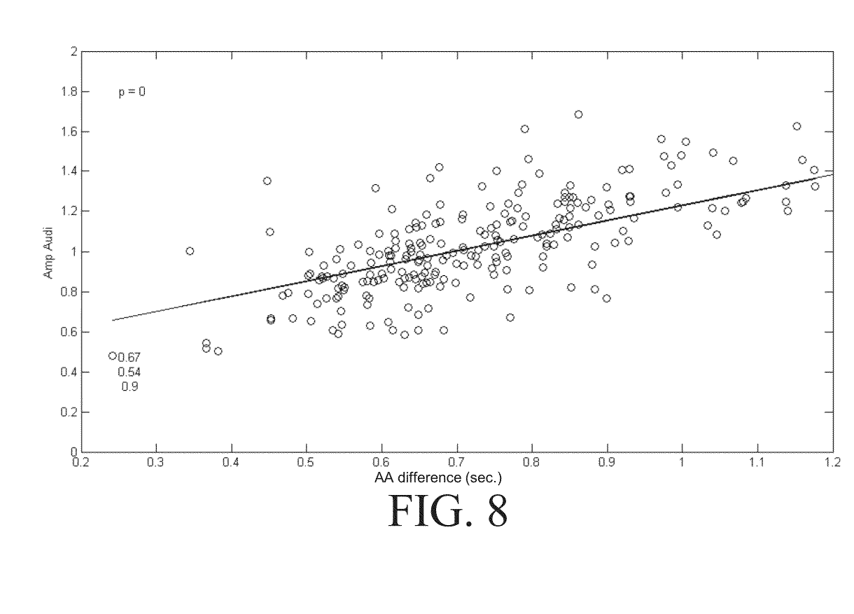

Atrial fibrillation is an abnormal heart rhythm, which is caused by irregular and uncoordinated beatings between the atria and the ventricles. FIG. 8 shows a scatter diagram which indicates a patient with atrial fibrillation. As shown in FIG. 8, the AA differences between adjacent waveform intervals cannot be grouped into categories or clusters. Such condition is identified in atrial fibrillation patients. Therefore, scattered AA differences in a scatter diagram indicates an atrial fibrillation condition of a subject.

IV. Atrial Flutter

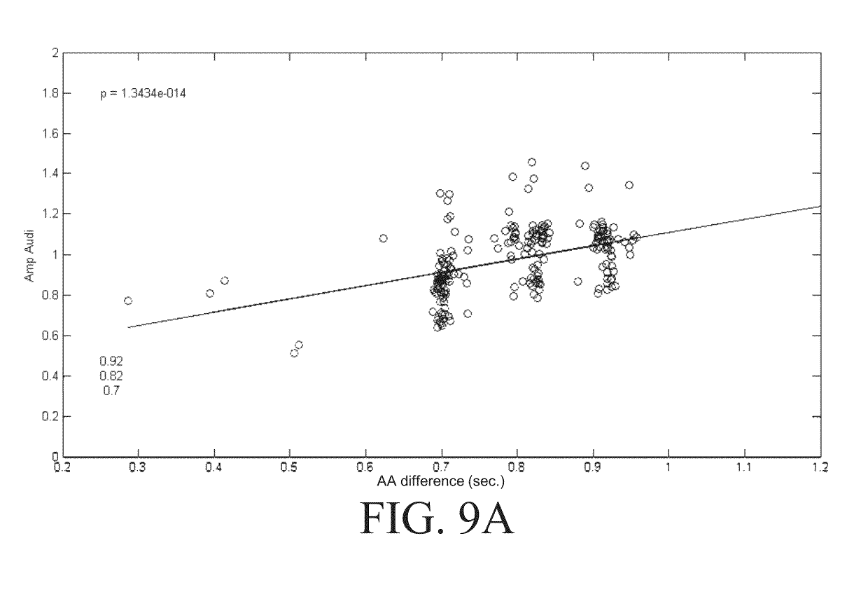

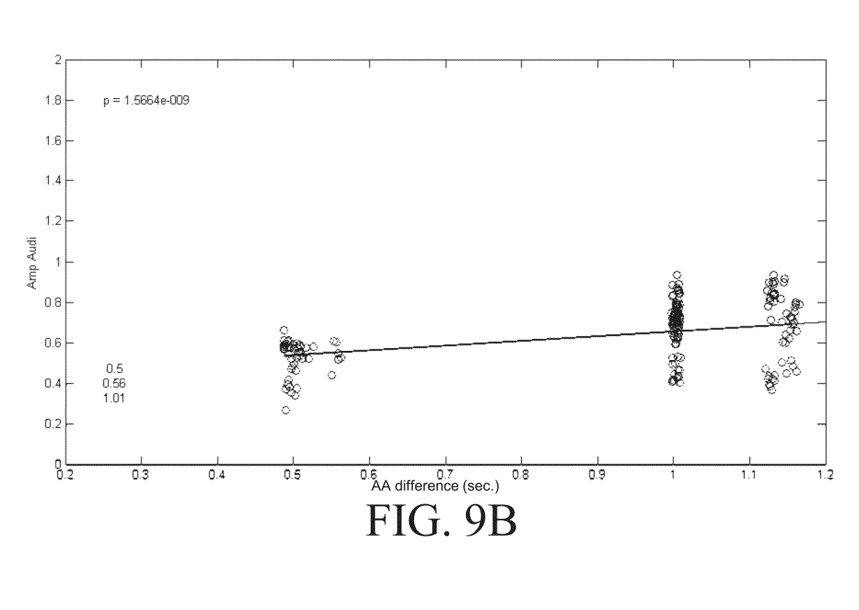

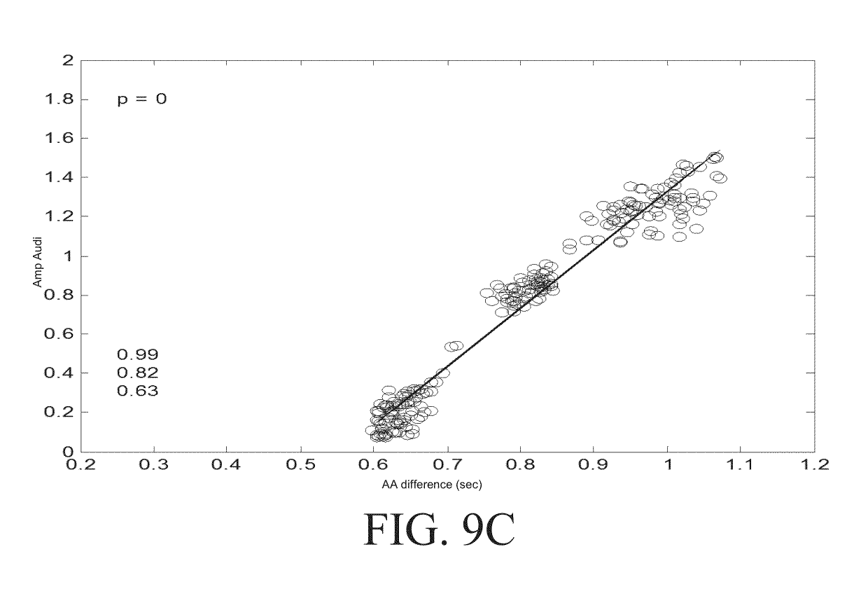

Atrial flutter is a kind of tachyarrhythmia characterized by atrial rates at about 240 to 400 beats per minutes. Atrial flutter can be identified in an electrocardiogram by sawtoothed P waves. Instead, as shown in FIG. 9A, a patient with atrial flutter was examined. The AA difference data points are clearly grouped into three categories, at around 0.7 sec, 0.84 sec and 0.94 sec. Three centroids are clearly separable and can be identified when perform a k-means clustering. Therefore, a scattered yet grouped AA differences in a scatter diagram indicates an atrial flutter condition of a subject.

V. Atrial Premature Contraction and Ventricular Premature Contraction

Premature contraction is a common heart arrhythmia. APC is characterized in abnormal and premature heartbeats originated from aria. The abnormal is generally caused by wrongful and irregular electrical signals generated from the sinus node in the upper chamber of the heart. APC disrupts normal and regular heart rhythm. When an APC event occurs, a premature P wave on an electrocardiogram can be spotted. The premature P wave comes up earlier than a non-APC P wave. Shown in FIG. 9B was a scatter diagram of a patient with APC. The AA difference data points are also clearly grouped into three categories. Three centroids are clearly separable and can be identified when perform a k-means clustering. Therefore, a scattered yet grouped AA difference in a scatter diagram also indicates an APC condition of a subject.

VPC is a relatively common event where the heartbeat is initiated by an abnormal heartbeat initiator. Single isolated VPC may be asymptomatic in healthy individuals and is hard to catch with conventional ECG devices. Shown in FIG. 9C was a scatter diagram of a patient with VPC. The AA difference data points are also clearly grouped into three categories. Three centroids are clearly separable and can be identified when perform a k-means clustering. Therefore, a scattered yet grouped AA differences in a scatter diagram also indicates a VPC condition of a subject.

As disclosed in both FIGS. 9A, 9B and 9C, grouped AA differences in the scatter diagram may indicate that a patient have atrial flutter, APC/VPC or a combination syndrome.

VI Pulse Deficit

Pulse deficit is the difference between an apical pulse and a peripheral pulse. It is a characteristic of several arrhythmias that cannot be observed by using ECG device only. FIG. 6 shows an electrocardiogram (6-1) and an audiocardiovasculogram (6-2) of a patient with VPC. 6-1 and 6-2 are respectively derived from an ECG probe and an ACVG sensing device. The signals received from both the ECG probe and the ACVG are synchronized and the graphics are aligned. As shown in FIG. 6, there was a VPC captured by ECG probe between 20^{th} sec. and 21^{st} sec. in 6-1. In 6-2, the pulse approximately starts from 20.5^{th} sec. presents a reduced peak Y value (Y value of A1) in the first wave followed by a prolonged AA difference (interval 602) and an augmentation (amplitude enhancement) in the next pulse (Y value of A2). Therefore, a reduced peak Y value in the first wave from a waveform data indicates a pulse deficit occurs in a subject

II. Blood Pressure

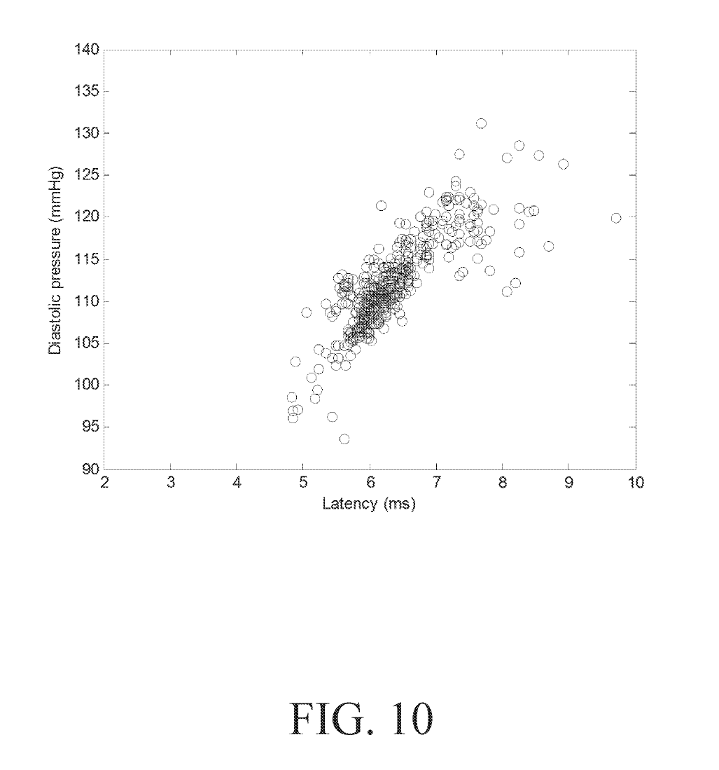

Blood pressure is a measurement of the force applied to the vessel wall of arteries. It is a common parameter to indicate many cardiovascular diseases alone or in combination with other parameters. FIG. 10 shows a scatter diagram which indicates a healthy individual. Both ACVG sensing device and ECG probe are used to give FIG. 10. ACVG sensing device and ECG probe are equipped on a subject and signals are synchronized. The measurement of horizontal axis of the scatter diagram is Latency which is an AR interval. The AR interval is the time interval (in horizontal axis) between one data point from ACVG and one data point from ECG signals. In one example, the AR interval is the distance of X value of R point in an electrocardiogram and X value of A point in an audiocardiovasculogram synchronized with said electrocardiogram (interval 601 as shown in FIG. 6). In one example, the two data points are each determined by identifying the point with a peak Y value within 0.5 second interval in ACVG and ECG signals. The measurement of vertical axis is the diastolic pressure of a patient. As shown in FIG. 10, the AR interval is in positive correlation with diastolic pressure. Therefore, the AR interval can represents the blood pressure of a subject.

VIII. Complete Left Bundle Branch Block

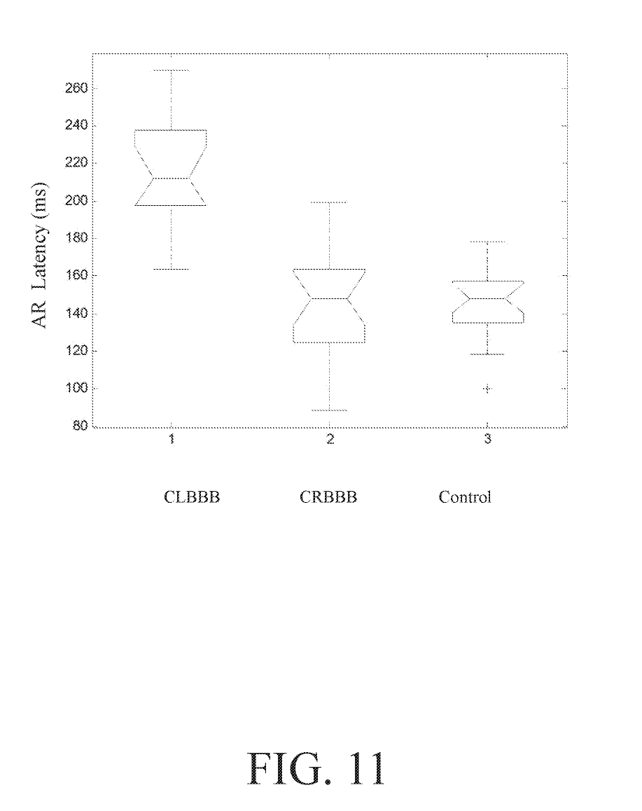

In healthy individual, the contraction of left ventricle and right ventricle occur approximately in the same time. CLBBB is a conduction obstruction or delay of an electrical impulse signal to induce left ventricle contraction and therefore lead to a delayed contraction than the right ventricle. It can be identified by wider QRS complexes with abnormal V1 and V6 on an electrocardiogram. As contraction of the left ventricle contributes the peripheral pulse, measuring the latency between peripheral pulse and heartbeat can indicate the delay of left ventricle contraction. Shown in FIG. 11 was a boxplot of patients with CLBBB, patients with CRBBB (complete right bundle branch block), and healthy individuals (Control). The measurement of vertical axis of the boxpolt is AR latency (AR interval). As shown in FIG. 11, patients with CLBBB have a higher mean of AR latency (AR interval) than healthy individuals and patients with CRBBB. The mean of AR latency in CLBBB patients is approximately the mean of AR latency in healthy individuals plus 1.5- to 2-fold of standard deviation. Therefore, a higher mean of AR intervals indicates a CLBBB condition of a subject.

IX. Ejection Fraction (EF)

EF is a fraction of blood pumped out from the heart with a heartbeat. A normal EF of left ventricle is over 50 percent (stroke volume pumped out of the ventricle divided by total amount of blood in the left ventricle). It is a common parameter to indicate a subject's heart disease, such as heart failure, and commonly measured by echocardiography. FIG. 4B shows a typical ACVG waveform which can indicate the status of a ventricular ejection of left ventricle. The ventricular ejection can be divided into two phases, a rapid ejection phase followed by a reduced ejection phase. A rapid ejection phase occurs when the pressure in ventricle is higher than artery during ventricular contraction. In this phase, cardiac cycle reaches a maximum ejection rate. After rapid ejection phase, the pressure in ventricle is reduced and the cardiac cycle enters a reduced ejection phase. In this phase, the blood is pumped by inertia and the ejection rate slows down. For the reason that peripheral pulse is mainly contributed by contraction of left ventricle, the rapid ejection phase and the reduced ejection phase of left ventricle can be presented by an audiocardiovasculogram, as shown in the first wave 401 and the second wave 402 in the ACVG waveform of FIG. 4B respectively. Therefore, the interval of the first wave 401 in an ACVG waveform can represent the ejection fraction of left ventricle within a heartbeat.

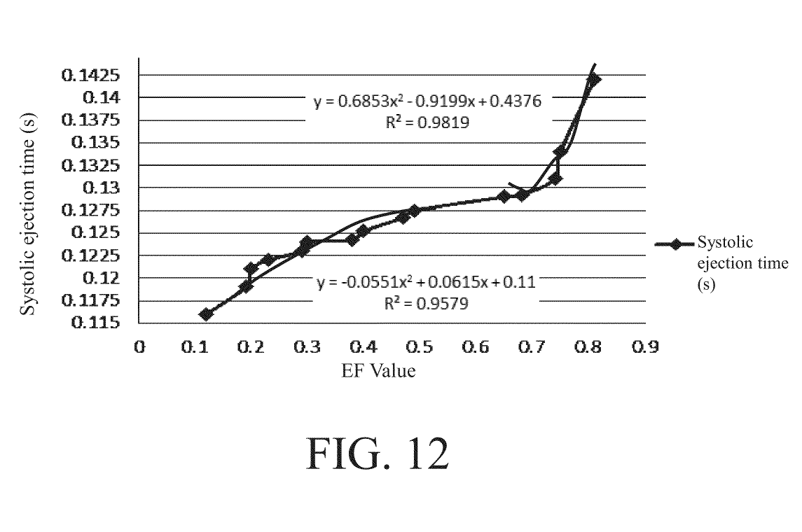

FIG. 12 shows a curve diagram which indicates the correlation between interval of the first wave 401 (systolic ejection time) and EF value. The measurement of vertical axis of the curve diagram is systolic ejection time (sec.) which was measured by ACVG sensing device. The measurement of horizontal axis of the curve diagram is EF value which was measured by echocardiography. As shown in FIG. 12, there are fifteen individuals were included in the experiment. Five out of the fifteen were healthy individuals with EF value over 65%, who also have longer systolic ejection time. The remaining ten individuals were patients with heart failure, who have EF value less than 50%. The less EF value also result in shorter systolic ejection time, as shown in FIG. 12. Therefore, the interval of the first wave in an ACVG waveform indicates the EF value of left ventricle of a subject.

What is claimed is:

1. An audiocardiovasculography (ACVG) sensing device, comprising
an ACVG sensor comprising a configured to sense signals of heart beat and arterial pulse in a predetermined period and to transform the sensed signals to electrical output; and
an analog-to-digital converter configured to receive the electrical output and to convert the electrical output into digital signals.

2. The ACVG sensing device according to claim 1, wherein the ACVG sensor comprises a capacitive microphone, and wherein the electrical output is C-ACVG.

3. The ACVG sensing device according to claim 2, wherein the capacitive microphone comprises a housing and a diaphragm, wherein the housing and the diaphragm define a medium cavity.

4. The ACVG sensing device according to claim 1, wherein the ACVG sensor has a response frequency at least including a range from approximately 0.5 hertz to approximately 1000 hertz.

5. The ACVG sensing device according to claim 1, wherein the ACVG sensor comprises a piezoelectric microphone or a blood pressure monitor, and wherein the ACVG sensing device further comprises a processor capable to calculate a derivative from the digital signals.

6. A method for sensing ACVG using the ACVG sensor of claim 1, comprising
sensing signals of heart beat and arterial pulse in a predetermined period;
transforming the sensed signals to electrical output; and
converting the electrical output into digital signals.

7. The method according to claim 6, wherein the ACVG sensor comprises a capacitive microphone; and wherein the electrical output is C-ACVG.

8. The method according to claim 6, wherein the capacitive microphone comprises a housing and a diaphragm, wherein the housing and the diaphragm define a medium cavity.

9. The method according to claim 6, wherein the ACVG sensor has a response frequency at least including a range from approximately 0.5 hertz to approximately 1000 hertz.

10. The method according to claim 6, further comprising calculating a derivative from the digital signals, wherein the ACVG sensor comprises a piezoelectric microphone or a blood pressure monitor.

11. A method of determining a physiological condition, comprising
receiving an ACVG;
providing a waveform data by processing the ACVG;
extracting at least one data point from a predetermined interval of the waveform data;
obtaining at least one indicator based on the at least one data point;
determining a physiological condition according to the at least one indicator.

12. The method according to claim 11, wherein processing the ACVG comprises rectifying the ACVG to obtain a rectified ACVG, filtering the rectified ACVG to obtain a filtered ACVG, and collecting a series of filtered ACVG to generate a waveform data.

13. The method according to claim 12, wherein the rectified ACVG is filtered through a zero phase shift bandpass filter.

14. The method according to claim 13, wherein the zero phase shift bandpass filter has a response frequency at 5 to 35 Hz.

15. The method according to claim 11, wherein extracting at least one data point from a predetermined interval of the waveform data comprises differentiating the waveform data to obtain a differentiated waveform data; and removing, within the differentiated waveform data, a data having a target response frequency range to obtain a pre-convolution waveform data.

16. The method according to claim 15, the target response frequency range is at above 30 Hz.

17. The method according to claim 11, wherein the at least one data point has a peak Y value with a corresponding X value within a predetermined time interval.

18. The method according to claim 17, the peak Y value is defined by convoluting the pre-convolution waveform data to obtain a convolution waveform data.

19. The method according to claim 17, wherein the predetermined time interval is 0.5 second interval.

20. The method according to claim 11, wherein the physiological condition is a cardiovascular-related condition.

21. The method according to claim 20, wherein the at least one indicator is a series of AA differences, and wherein the cardiovascular-related condition is selected from pacemaker-dependent condition, atrial fibrillation, atrial flutter, APC, and VPC.

22. The method according to claim 20, wherein the at least one indicator is a peak Y value of the first wave in a waveform data; wherein the cardiovascular-related condition is pulse deficit.

23. The method according to claim 20, wherein the at least one indicator is the interval of the first wave in a waveform data; wherein the cardiovascular-related condition is ejection fraction.

24. The method according to claim 11, further comprising receiving an ECG data; and generating an ECG waveform data which is synchronized with the waveform data derived from ACVG.

25. The method according to claim 24, wherein the at least one indicator is a series of AR intervals, and wherein the physiological condition is selected from blood pressure and CLBBB.
